(19) United States
(12) Patent Application Publication
(10) Pub. No.: US 2015/0313751 A1
(43) Pub. Date: Nov. 5, 2015

(54) MANDIBULAR ADVANCEMENT DEVICE

(71) Applicant: SleepWell Enjoy Life Limited, Sheffield (GB)

(72) Inventor: Jeremy Kenyon, Sheffield (GB)

(21) Appl. No.: 14/402,312

(22) PCT Filed: May 24, 2013
(86) PCT No.: PCT/GB2013/051379

§ 371 Date: Nov. 19, 2014

(30) Foreign Application Priority Data
May 28, 2012 (GB) 1209406.6

(51) Int. Cl. A61F 5/56; A61C 9/00

(52) U.S. Cl. CPC A61F 5/566; A61C 9/0006

(57) ABSTRACT

A mandibular advancement device having an upper retainer positionable in front of teeth of a user's upper jaw and connected to a lower retainer positionable behind teeth of a user's lower jaw. The lower retainer comprises a support structure towards the rear of the lower retainer. The support structure has a shape and/or is made from a material such that, in use, a relatively high force is applied to two or more rearward teeth of the lower jaw to move the lower jaw forwards, and a relief region towards the front of the lower retainer configured to, in use, apply a relatively low force on one or more forward teeth of the lower jaw.

### FIELD OF INVENTION

The present invention relates to a mandibular advancement device and/or a method of manufacturing a mandibular advancement device.

### BACKGROUND

When a person is sleeping or anesthetised, for example, their jaw can move backwards as their muscles relax. This backwards movement of their jaw causes the soft tissues at the back of the jaw to move towards the back of their throat. This movement of the soft tissue restricts or in some cases blocks the airways which can cause problems such as snoring, sleep apnea, or reduced blood oxygenation during anaesthesia.

One technique for avoiding or reducing problems of snoring, obstructive sleep apnea and/or reduced blood oxygenation during anaesthesia is mandibular advancement, i.e. holding a person's lower jaw in a forwards position, compared to a natural rest position. Holding the lower jaw in a forwards position holds the soft tissue away from the back of a person's throat to increase the flow of air through the airway passage.

Often a mandibular advancement device (MAD) is used to hold the lower jaw in the forwards position. There are three common types of devices used for this purpose: standard fit pre-moulded devices, i.e. devices sold as “off the shelf, ready to use devices”, self-fit moulded devices, i.e. devices bought “off the shelf”, but a user can mould the device to include an impression of a their teeth; and custom made devices, i.e. devices where a dentist moulds the device specifically for a user's teeth and needs.

A type of standard fit pre-mould device of the prior art is indicated at 110 in FIG. 1. The device has a lower and upper tray 112, 114. The lower tray 112 has a lower retainer 116 positionable behind teeth of a user's lower jaw, a cover portion 118 positionable in front of teeth of a user's lower jaw and a portion 120 extending between the retainer 116 and the cover portion 118 and positionable over teeth of a user's lower jaw. The upper tray 114 has an upper retainer 122 positionable in front of teeth of a user's upper jaw, a cover portion 124 positionable behind teeth of a user's upper jaw and a portion 126 extending between the retainer 122 and the cover portion 124 to be positionable over teeth of a user's upper jaw. A hinge 128 connects the upper and lower trays.

In use, the upper tray 114 is positioned over teeth on a person's upper jaw and the lower tray is positioned over teeth on a person's lower jaw. The upper retainer 122 of the upper tray provides an anchor point for the MAD, such that the lower retainer 116 can apply a force to teeth of a person's lower jaw to move their lower jaw forward. FIG. 2 shows a schematic profile of a lower retainer 116 of the prior art positioned behind teeth of a lower jaw.

It can be seen that in this example the lower retainer 116 has an arched shape substantially following the profile of a person's teeth and the lower retainer 116 is positioned adjacent the central incisors 130, lateral incisors 132, cuspid (or canine) 134, first 136 and second 138 biscupid (or pre-molars), and the first molar 140. Due to the material the MAD is manufactured from, typically a thermoplastic such as a polyvinyl resin, for example a vinyl acetate-ethylene copolymer such as poly(ethyl vinyl acetate), or a polyolefin such as polyethylene or polypropylene, the lower retainer 116 deforms so that the force to move the jaw forwards is primarily applied to the central 130 and lateral 132 incisors.

A self-fit moulded device works under a similar principle. However, the upper and lower trays of these devices each comprise an outer shell into which a moulding material for forming an impression of a user's teeth is positioned. Although, the self-fit moulded devices do not deform to the same extent as a standard fit pre-moulded device, they are still designed to apply the majority of the forward force on a person's central and lateral incisors.

It is believed in the industry that a retainer having a shape that follows the semi-ovular profile of the teeth is the most effective method of applying force to move the teeth forwards.

### SUMMARY OF INVENTION

The present invention seeks to overcome problems of mandibular advancement devices, including standard fit pre-moulded devices and individually moulded devices.

Accordingly, in a first aspect the invention provides a mandibular advancement device having an upper retainer positionable in front of teeth of a user's upper jaw and connected to a lower retainer positionable behind teeth of a user's lower jaw, the lower retainer comprising:
a support structure towards the rear of the lower retainer, the support structure having a shape and/or made from a material such that, in use, a relatively high force is applied to two or more rearward teeth of the lower jaw to move the lower jaw forwards; and
a relief region towards the front of the lower retainer configured to, in use, apply a relatively low force to one or more forward teeth of the lower jaw.

As discussed above, it is believed in the art that the shape of the retainer should substantially match the profile of the teeth, such that a force to move the jaw forward is applied to a user's forward-most teeth. The inventor of the present invention has departed from this prejudice in the art by providing a mandibular advancement device (MAD) configured to apply the majority of the force to move the lower jaw forwards to the more rearward teeth, instead of the more forward teeth. The present inventor has recognised that the rearward teeth can generally resist a greater force than the forwardmost teeth, thus a greater force can generally be applied to the rearward teeth without any undesired effects, so that the force applied to the rearward teeth as a magnitude in the forwards direction substantially equal to that required to move the jaws forwards.

User's of MADs of the prior art can suffer from discomfort due to the force applied to their forward teeth, and in some extreme cases user's of MADs of the prior art may be unable to continue use of the MAD because of high discomfort. The relief region means that a relatively low force, which in some embodiments equates to no force, is applied to one or more forward teeth. This reduction in applied force improves comfort for a user.

MADs of the prior art are currently designed to apply the majority of the forward pressure required to move a jaw forward to the lower jaw, predominately via a user's lower four front teeth (central and lateral incisors). The invention shifts some or all of the forward pressure onto the more rearward lower teeth. This change in pressure distribution can be achieved because the present inventor has recognised that applying a force substantially perpendicular to the line of the teeth of the more rearward canines and pre-molars can apply a force having the required magnitude in the forwards direction to move the lower jaw forwards.

The rearward teeth may be the lateral incisors, the cuspids (or canines), one or more of the bicupsids (or pre-molars) or one or more of the molars. The forward teeth may be one or more of the central incisors, or when the rearward teeth are rearward of the cuspids (or canines), the forward teeth may be one or more of the central incisors and the lateral incisors. For example, the relief structure may apply relatively low force to one, two, three or four of the forward teeth.

The relief region may be configured to, in use, apply no force on one or more forward teeth of the lower jaw.

In exemplary embodiments, the relief region has a relief structure shaped to, in use, reduce contact of the lower retainer with one or more forward teeth of a user's lower jaw.

In exemplary embodiments, the relief structure may be shaped to, in use, eliminate contact of the lower retainer with one or more forward teeth of a user's lower jaw.

In the present application, directions are defined with respect to a device when positioned in a user's mouth. A rearward-forward direction is defined as a direction into and out of a users mouth; an upper-lower direction is defined as a direction towards the upper and lower jaw of a user; and the right and left direction is defined as a direction transverse to the other two directions with the right side being the right hand side of a user's jaw from their own perspective.

The support structure may generally define a convex shape. The relief region may have a relief structure. The relief structure may comprise a convex shape. In such embodiments, the diameter of the convex relief structure may be greater than the diameter that would complete the semi-oval convex shape of the support structure in the region of the relief structure.

The relief region may have a relief structure, and the relief structure may comprise a linear section extending between two sides of the support structure. The linear section may extend substantially parallel to an axis transverse to both the upper-lower direction and the forward-rearward direction, i.e. in exemplary embodiments, the axis in the right-left direction.

The relief structure may comprise an arch curved towards the support structure. The support structure may be generally convex, and the relief structure may be generally concave. In such embodiments, in use, the relief structure may curve away from one or more of the forward teeth of a user's lower jaw.

The mandibular advancement device may comprise a more compliant material than the material of the support structure positioned in the relief region. For example, the more compliant material may be positioned on a front face of the relief structure for, in use, contacting one or more forward teeth of a user's lower jaw. For example, the more compliant material may be a gel. Alternatively, the relief structure may be manufactured from a more compliant material than the support structure.

The mandibular advancement device may be a standard fit pre-moulded device or may be a self-fit moulded device. A self-fit moulded device is a device configured to permit a user to form an impression of their teeth in the device. Self-fit devices are sold off the shelf, as opposed to custom fit devices that are fitted by dentists. Self-fit devices have more design constraints than a device fitted by a dentist because the self-fit devices need to be easy for someone without dental training and without access to special dental equipment to use.

In exemplary embodiments where the device comprises an impression of a user's teeth (e.g. a self-fit moulded device), a gap may be provided in the impression in a forward region of the retainer to form the relief region. That is, the gap may be located in a position corresponding to, in use, a location adjacent the central and/or lateral incisors. For example, a portion of the impression may be removed in a forward region of the retainer to form the relief region.

A more compliant material than the material of the impression may be provided in the gap, for example the material may be replaced with a gel. Reference to the compliance of the material of the impression refers to the material when set.

The support structure may have a thickness such that deformation of the support structure is limited so as to permit the support structure to exert a relatively high force on one or more of the rearward teeth.

Alternatively or additionally, the support structure may be made from a material such that deformation of the support structure is limited so as to permit the support structure to exert a relatively high force on one or more of the rearward teeth.

In exemplary embodiments, the support structure may comprise two arches. The remainder of the support structure may generally define a semi-oval shape in a forward-rearward direction of the retainer, and a diameter of each of the two arches may be less than a diameter of an arch that would complete the oval shape of the support structure. The intention of having such a diameter is that the arches have a diameter less than a diameter of a row of forward teeth of a lower jaw of a user. The two arches may be of substantially equal diameter such that the support structure is symmetrical in shape. The relief structure may adjoin the two arches.

The thickness of the retainer may increase from a rearward position to a forward position of the support structure. For example, the thickness of the retainer may increase such that the thickness is greatest in the region of the relief structure and apex of the arches of the support structure.

The upper retainer may form part of an upper tray and the lower retainer may form part of a lower tray. In such embodiments, the upper and lower trays may each comprise a portion postionable over respective teeth of a user's upper and lower jaw.

The lower tray may be open in a front region so as to, in use, not cover a front side of teeth on a user's lower jaw and/or the upper tray may be open in a rear region so as to, in use, not cover a rear side of teeth of a user's upper jaw. Leaving the front side of the lower teeth and rear side of the upper teeth uncovered can improve comfort for a user.

The upper and lower tray may be connected by a hinged joint, by one or more pins, one or more clips or may be formed as a single component, for example the upper and lower tray may be co-moulded. In exemplary embodiments, the upper and lower retainer may be formed as a single component, for example the upper and lower retainer may be co-moulded.

In exemplary embodiments, in use, the upper retainer is positioned to be substantially above the lower retainer;
and the upper retainer and the lower retainer each have a generally semi-oval shape, and
the lower retainer is profiled such that in use, at least a portion of the lower retainer is profiled to protrude more radially outwards from the generally semi-oval shape of the lower retainer at an end of the lower retainer furthest from the upper retainer than an end nearest the upper retainer; and/or
the upper retainer is profiled such that in use, at least a portion of the upper retainer is profiled to protrude more radially inwards from the generally semi-oval shape of the upper retainer at an end of the upper retainer furthest the lower retainer than an end nearest the lower retainer.

In a second aspect of the invention there is provided a mandibular advancement device having an upper retainer positionable in front of teeth of a user's upper jaw and connected to a lower retainer positionable behind teeth of a user's lower jaw, such that, in use, the upper retainer is positioned to be substantially above the lower retainer;
wherein the upper retainer and the lower retainer each have a generally semi-oval shape, and
the lower retainer is profiled such that in use, at least a portion of the lower retainer is profiled to protrude more radially outwards from the generally semi-oval shape of the lower retainer at an end of the lower retainer furthest from the upper retainer than an end nearest the upper retainer; and/or
the upper retainer is profiled such that in use, at least a portion of the upper retainer is profiled to protrude more radially inwards from the generally semi-oval shape of the upper retainer at an end of the upper retainer furthest the lower retainer than an end nearest the lower retainer.

Standard-fit mandibular advancement devices (MADs) of the prior art have a lower and upper retainer profiled to follow the profile or general form of a user's teeth. There is a general belief in the industry that a profile following the profile or general form of a user's teeth improves comfort for a user. However, the inventor of the present invention has recognised that in fact comfort for a user can be improved if the lower and/or upper retainer protrudes more at a location corresponding to a base of the teeth than at a location corresponding to a tip of the teeth. In addition, applying the force at the base of the teeth reduces the tendency of the MAD to slip off the teeth compared to MADs of the prior art. When a user wears a MAD of the prior art, the force applied by the lower retainer to move the lower jaw forwards and/or the anchoring force applied by the upper retainer is applied to the tips, or close to the tips of the teeth. This means there is a large moment applied to a tooth about its root. By providing a profile that protrudes more in a location corresponding to the base of a tooth, the force applied by the upper and/or lower retainer is applied at a base of a tooth which is closer to the end of the tooth root, which reduces the moment applied to each root. Reduced moment means improved comfort and significantly reduces the likelihood of a user not being able to use the MAD due to high levels of discomfort.

MADs of the prior art place pressure over the tips of a user's teeth (i.e. at a position furthest from a user's gums). This is the case of standard fit pre-moulded devices and self-fit moulded devices. In individually moulded devices the comparatively more flexible nature of the impression material of individually moulded devices and the geometry of the MAD means that the pressure is generally shifted towards the tips of the teeth. The present invention instead shifts the pressure towards the base of the teeth, thus reducing the turning moment on the teeth, which increases comfort for a user.

The mandibular advancement device may be a pre-moulded standard fit mandibular advancement device.

The lower retainer and/or upper retainer may be curved from a position of minimal radial protrusion (outwards or inwards as applicable) to a position of maximum radial protrusion (outwards or inwards as applicable). This curved profile means that the upper and/or lower retainer is curved towards a location corresponding to an end of one or more teeth nearer a user's gum in a region of intended force application.

The lower retainer and/or upper retainer may be angled from a position of minimal radial protrusion (outwards or inwards as applicable) to a position of maximum radial protrusion (outwards or inwards as applicable). This angled profile of the lower retainer and/or upper retainer means that the retainer is angled towards a location corresponding to an end of one or more teeth nearer a user's gum in a region of intended force application.

The lower and/or upper retainer may comprise a radially protruding rim (the rim protruding radially outwards or inwards as applicable). This means that a rim is provided in a region corresponding to an end of one or more teeth nearer a user's gum. The rim of the lower and/or upper retainer may be rounded.

The lower or upper retainer may be profiled to radially protrude between and inclusive of approximately 2 and 5 mm. The lower or upper retainer may be profiled to radially protrude between and inclusive of approximately 3 and 5 mm. The lower or upper retainer may be profiled to protrude approximately 3.5 mm, 4 mm or 4.5 mm.

In a third aspect of the present invention there is provided a mandibular advancement device having an upper retainer positionable in front of teeth of a user's upper jaw and connected to a lower retainer positionable behind teeth of a user's lower jaw, wherein
the lower retainer is profiled such that, in use, a region of the lower retainer intended to apply a force to move the jaw forwards is profiled so as to, in use, apply increased force in a location corresponding to an end of one or more teeth nearer a user's lower gum than a location corresponding to an end of one or more teeth furthest from a user's lower gum; and/or
the upper retainer is profiled such that, in use, a region of the upper retainer intended to apply a force to anchor the device in a user's mouth is profiled so as to, in use, apply increased force in a location corresponding to an end of one or more teeth nearer a user's upper gum than a location corresponding to an end of one or more teeth furthest from a user's upper gum.

In a fourth aspect of the present invention there is provided a method of manufacturing a mandibular advancement device of the first aspect, second aspect, and/or third aspect.

The mandibular advancement device may comprise an impression of a user's teeth, and the method may comprise:
forming an impression of teeth of a user's lower jaw; and
removing a portion of the impression in a forwards region of the impression to form the relief structure.

Alternatively, the mandibular advancement device may comprise an impression of a user's teeth, and the method may comprise:
forming an impression of only the rearward teeth of a user's lower jaw.

The method may comprise forming the impression of the teeth in a casing.

### DESCRIPTION OF DRAWINGS

Embodiments of the invention will now be described with reference to the accompanying drawings, in which:

FIG. 1 shows a perspective view of a mandibular advancement device of the prior art;

FIG. 2 shows schematically a plan view of a lower retainer of a mandibular advancement device of the prior art positioned in a user's mouth;

FIG. 3 shows a perspective view of a mandibular advancement device according to an embodiment of the present invention;

FIG. 4 shows a plan view of the mandibular advancement device shown in FIG. 3;

FIG. 5 shows a schematically a plan view of a lower retainer of a mandibular advancement device according to an embodiment of the present invention;

FIG. 6 shows schematically a plan view of a mandibular advancement device according to a further embodiment of the present invention; and

FIG. 7 shows schematically a cross-section of a mandibular advancement device according to another embodiment of the present invention.

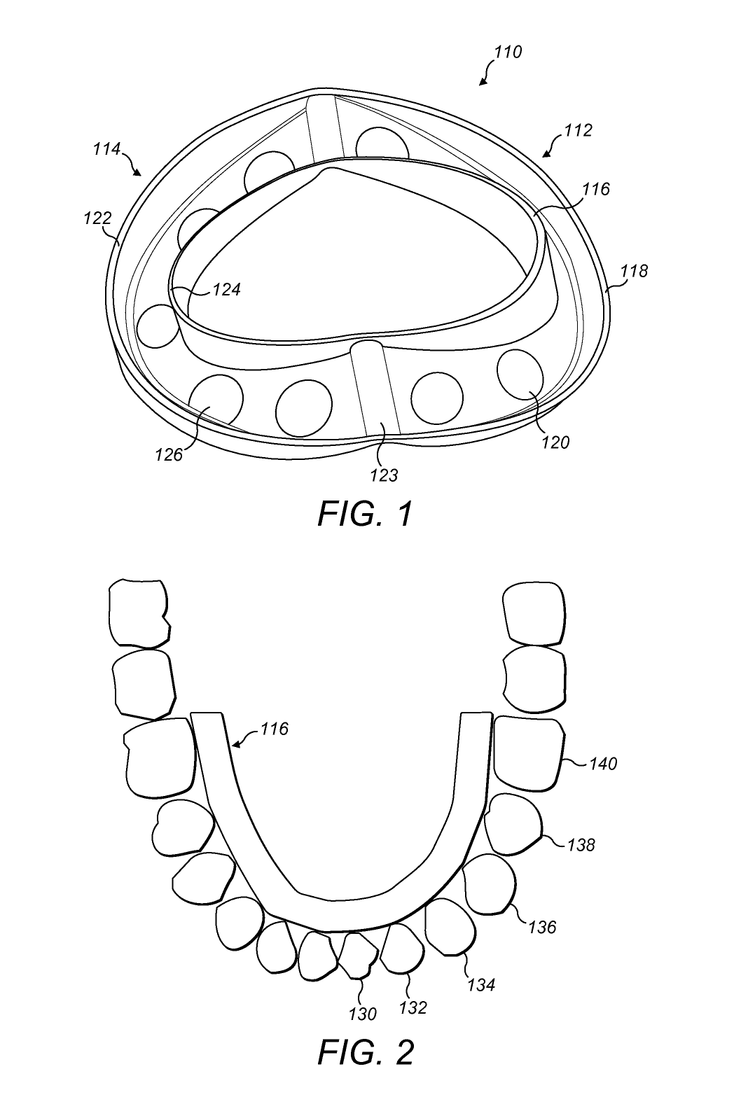

### DETAILED DESCRIPTION

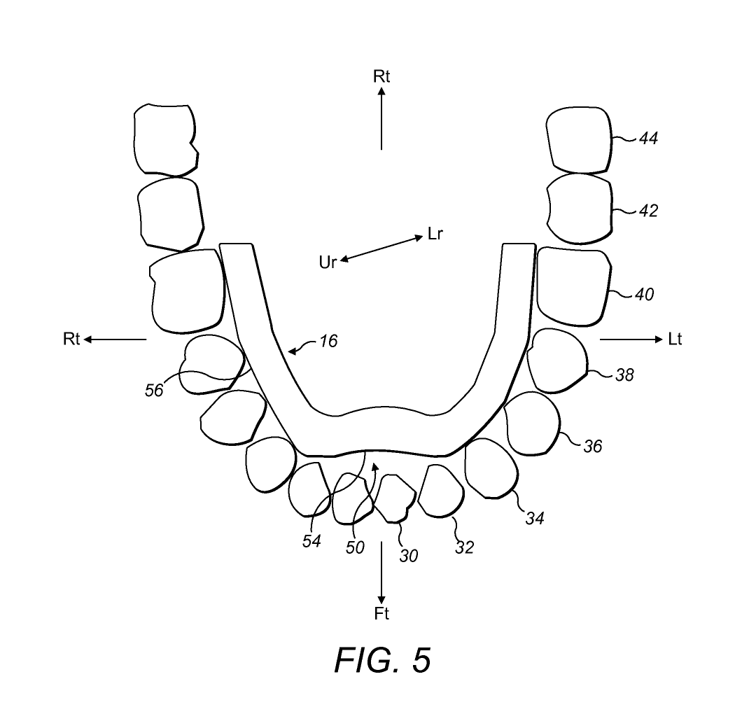

In the following description, directions are defined with respect to a device when positioned in a user's mouth. A rearward Rr direction is defined as a direction into a users mouth, a forward Fd direction is defined as a direction out of a users mouth, an upper Ur direction is defined as a direction towards the upper jaw of a user from the lower jaw, a lower Lr direction is defined as a direction towards the lower jaw of a user from the upper jaw, and a right Rt and left Lt direction is defined as a direction transverse to the other two directions with the right side being the right hand side of a user's jaw from their own perspective, arrows indicating the directions are shown in FIG. 5.

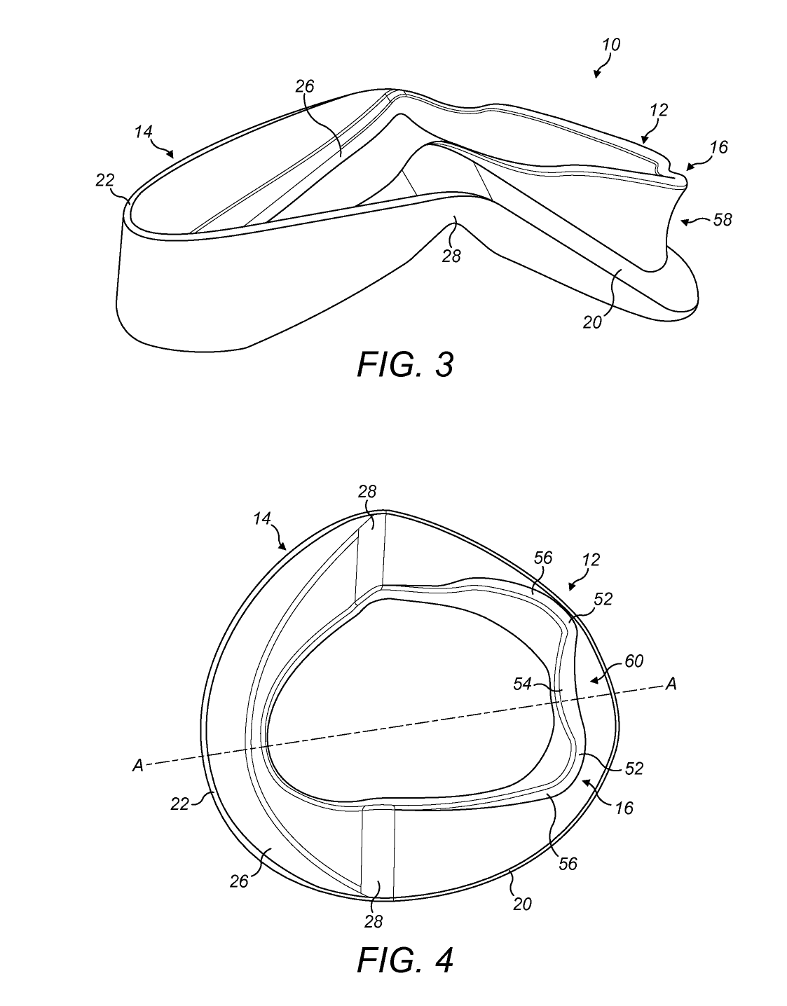

Referring to FIGS. 3 and 4, a mandibular advancement device (MAD) is indicated generally at 10. In this embodiment the MAD is a standard-fit pre-moulded MAD. The MAD has a two generally semi-oval trays connected to form a generally oval ring. The generally oval ring can be folded to permit an upper tray 14 of the MAD to be positioned on a user's upper teeth and a lower tray 12 of the MAD to be positioned on a user's lower teeth.

The lower tray 12 has a retainer 16 for positioning behind teeth of a user's lower jaw. The retainer 16 has a semi-oval shape and is positioned on an inner circumference of the lower tray. A portion 20 is adjoined to the retainer 16 positioned transverse to the retainer 16 such that in use the portion 20 is positioned over the tips of a user's teeth.

The upper tray 14 has a retainer 22 for positioning in front of teeth of a user's lower jaw. The retainer 22 has a semi-oval shape and is positioned on an outer circumference of the upper tray. A portion 26 is adjoined to the retainer 22 positioned transverse to the retainer 22 such that in use the portion 26 is positioned over the tips of a user's teeth.

A hinge 28 joins the upper tray 14 and the lower tray 12. The hinge 28 permits the MAD to fold such that in use the upper tray 14 can be positioned substantially above the lower tray 12. The hinge 28 is a live resilient hinge, such that when not in use, the rest state of the hinge is such that the upper tray is substantially adjacent the lower tray rather than being above the lower tray (i.e. the position shown in FIGS. 3 and 4). This means that in use, the hinge is resilient to permit the upper tray to be positioned above the lower tray. Such resilience can help to maintain the correct positioning of the tray with respect to a user's teeth.

Referring to FIG. 5, the retainer 16 is schematically shown positioned adjacent a user's lower teeth. In this embodiment, the retainer 16 is dimensioned to be positioned in a region associated with a user's central incisors 30, lateral incisors 32, cuspids 34, bicuspids 36 and 38, and the first molar 40. However, in other embodiments the retainer 16 may extend to a position in a region associated with the second 42 and/or third 44 molars or it may extend to a lesser extent so as not to be in a region associated with the first molar 40.

The retainer 16 has a support structure 56 positioned towards the rear of the retainer such that in this embodiment the support structure is adjacent the cuspids 36, bicuspids 38 and first molar 40. In alternative embodiments the support structure may also be adjacent the lateral incisors 34, the second molar 42 and/or the third molar 44.

A relief structure 54 is positioned towards the front of the retainer 16. In this embodiment, the relief structure 54 is in a region of the central 30 and lateral 32 incisors. However, in other embodiments the relief structure 54 may be in a region of only the central incisors 30 or alternatively in a region of the central 30 and lateral 32 incisors and the cuspids 34. The relief structure 54 is profiled to form a gap 50 between the retainer 16 and the central 30 and lateral 32 incisors when the relief structure 54 is positioned in a user's mouth.

Referring again to FIGS. 3 and 4, said gap 50 is formed by the relief structure 54 curving away from a region corresponding to a user's forward teeth, i.e. curving away from the front of the MAD.

The support structure 56 extends from a position near the hinge 28 to adjoin the relief structure 54. In this embodiment the support structure 56 is symmetrical about an axis A in the forwards-rearwards direction. The support structure 56 forms two arches 52 towards the front of the lower tray 12. The relief structure 54 adjoins the two arches 52. The support structure 28 and the relief structure 54 form a contiguous single moulding.

The wall thickness of the retainer 16 increases from a position near the hinge 28 to a maximum at the relief structure 54 and the apex of the arches 52. The double arched shape and the increase in thickness of the retainer 16 towards the front of the retainer provides a structure that, in use, prevents the relief structure from contacting the central incisors 30 and prevents or substantially limits contact of the relief structure 54 with the lateral incisors 32.

Referring to FIG. 3, the lower retainer 16 on an outer side of the lower tray 12, i.e. a side that in use is positioned adjacent a user's teeth, has a curved profile 58. The curved profile is such that the retainer 16 curves towards a lowermost position of the lower tray 12, such that in use, the retainer curves towards an end of a user's teeth nearest to a user's lower gum.

The upper retainer 22 on an inner side of the upper tray 14, i.e. a side that in use is positioned adjacent a user's teeth, is angled towards an uppermost position of the upper tray. Such an angle means that in use the retainer is angled towards an end of a user's teeth nearest a user's upper gum.

In this embodiment the curved profile and the angle is dimensioned such that there is a 3 to 5 mm protrusion in the region corresponding to an end of a user's teeth nearest a user's gum. Referring to FIG. 7, in alternative embodiments, a rim 64c, 66c may be provided as an alternative to a curved or angled profile. In further alternative embodiments, the lower retainer may be angled and/or the upper retainer may have a curved profile.

The MAD may be made from a thermoplastic. For example, the MAD may be made from a polyvinyl resin, for example a vinyl acetate-ethylene copolymer such as poly(ethyl vinyl acetate), or a polyolefin such as polyethylene or polypropylene. In alternative embodiments any suitable material may be used, and it will be clear to a person skilled in the art which materials are suitable.

The MAD may be manufactured by a moulding process.

In use, the MAD 10 is placed in a user's mouth. The upper tray 14 is positioned over teeth of a user's upper jaw and the lower tray 12 is positioned over teeth of a user's lower jaw. The upper retainer 22 is positioned in front of teeth of a user's upper jaw and the lower retainer 16 is positioned behind teeth of a user's lower jaw.

The upper retainer 22 anchors the upper tray 14 in position. The angled profile of the upper retainer 22 means that the majority of the anchoring force is applied to a region of a user's teeth close to a user's upper gum.

The lower retainer 16 applies a force, in an opposite direction to the anchor force provided by the upper retainer, to move the jaw forwards. Due to the curved profile of the lower retainer, the majority of the force is applied at an end of a user's teeth nearest to a user's lower gums. Moving the jaw forwards, opens a user's airways and can alleviate snoring or sleep apnea or can maintain an open airway for surgical purposes.

The configuration of the support structure 56 and the relief structure 54 means that the force to move the lower jaw forwards is applied to a user's more rearward teeth and, in this embodiment, no force is applied to the central incisors and no or limited force is applied to the lateral incisors 32. This is achieved by positioning the support structure adjacent the more rearward teeth of a user's lower jaw and also profiling the support structure 56 and the relief structure 54 to have a rigidity such that the relief structure is substantially restricted from deforming towards a user's incisors.

The construction of the MAD 10 described improves comfort for a user, by removing/reducing the force applied, in use, to the central and lateral incisors. The curved profile of the lower retainer and the angled profile of the upper retainer further improves comfort for a user by applying a force to a user's teeth at a position closer to the user's gum line than MADs of the prior art, which reduces the torque applied to a user's teeth. Further, the curved profile of the lower retainer and the angled profile of the upper retainer helps the MAD stay in place and alleviates the problem associated with MADs of the prior art of the MAD having a tendency to slip off the teeth while in use.

Although the invention has been described above with reference to one or more preferred embodiments, it will be appreciated that various changes or modifications may be made without departing from the scope of the invention as defined in the appended claims.

For example, the relief structure may have an alternative shape, such as a linear profile e.g. extending linearly in the right-left direction from one side of the support structure to the other. Alternatively, the relief structure may be curved, such that in use the relief structure curves towards a user's teeth. However, in such embodiments the radius of such a curve is intended to be greater than the radius formed by a row of a user's central and lateral incisors, to eliminate or reduce contact of the relief structure with a user's teeth.

In alternative embodiments, the rigidity of the support structure required to apply a force to the rearward teeth and restrict the deformation of the relief structure may be provided by using a more rigid material, or a composite including a more rigid material, or alternatively or additionally by increasing the thickness of the support structure in one or more desired locations, without the need for the specific double arch structure disclosed in the above embodiment.

In further alternative embodiments, a material more compliant that the support structure may be provided in front of the relief structure. The more compliant material may in some embodiments contact a user's forward-most teeth, but the compliant nature of the material means that a much reduced force is transmitted to a users' forward-most teeth than in the MADs of the prior art. A suitable compliant material would be a gel. In other alternative embodiments, the relief structure may be manufactured from a material more compliant than the material of the support structure.

In another alternative embodiment the upper and lower tray may be connected by one or more pins or clips. In a further alternative embodiment, the upper and lower retainer may be provided as a single co-moulded component, such that the upper retainer is positionable in front of a user's upper teeth and a lower retainer protrudes from a lower end of the upper retainer and is positionable behind a user's lower teeth.

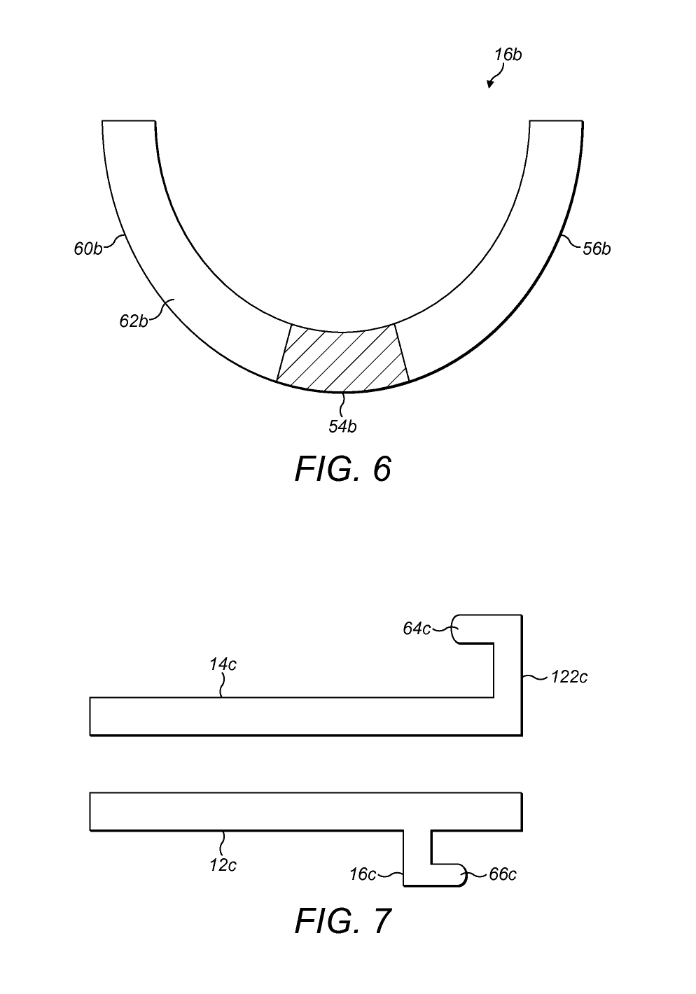

Yet a further alternative embodiment is illustrated schematically in FIG. 6. In this embodiment, the MAD is a self-fit device that is moulded to the shape of a user's teeth, and as such comprises an outer case 60b housing an impression 62b of a users' teeth. The construction of the outer case and the material of the impression is similar to that well known in the art so will not be explained in further detail here. In this embodiment, the casing and the impression form a support structure 56b, which in this embodiment is in use positioned behind a user's teeth, and over a user's teeth. A relief structure 54b is provided by providing a gap in the impression material in a region corresponding to one or more of the central incisors, or one or more of the central and lateral incisors, as desired. This construction achieves similar advantages as the construction of the standard fit device described above.

The gap in the impression may be formed by only forming an impression of a user's rearward teeth, or alternatively the gap in the impression may be formed by forming the impression of the teeth and then removing the desired portion of the impression.

1. A mandibular advancement device having an upper retainer positionable in front of teeth of a user's upper jaw and connected to a lower retainer positionable behind teeth of a user's lower jaw, the lower retainer comprising:
a support structure towards the rear of the lower retainer, the support structure having a shape and/or made from a material such that, in use, a relatively high force is applied to two or more rearward teeth of the lower jaw to move the lower jaw forwards; and
a relief region towards the front of the lower retainer configured to, in use, apply a relatively low force on one or more forward teeth of the lower jaw.

2. The mandibular advancement device according to claim 1, wherein the relief region is configured to, in use, apply no force on one or more forward teeth of the lower jaw.

3. The mandibular advancement device according to claim 1, wherein the relief region has a relief structure shaped to, in use, reduce contact of the lower retainer with one or more forward teeth of a user's lower jaw, or alternatively wherein the relief region has a relief structure shaped to, in use, eliminate contact of the lower retainer with one or more forward teeth of a user's lower jaw.

4. The mandibular advancement device according to claim 1, wherein the support structure generally defines a convex shape, and the relief region has a relief structure that generally defines a concave shape.

5. The mandibular advancement device according to claim 1, wherein the device is standard fit pre-moulded device or a self-fit moulded device comprising an impression of a user's teeth.

6. The mandibular advancement device according to claim 1, wherein the support structure has a thickness such that deformation of the support structure is limited so as to permit the support structure to exert a force on one or more of the rearward teeth, and/or wherein the support structure is made from a material such that deformation of the support structure is limited so as to permit the support structure to exert a force on one or more of the rearward teeth.

7. The mandibular advancement device according to claim 1, wherein the support structure comprises two arches, optionally wherein the two arches are of substantially equal diameter such that the support structure is substantially symmetrical in shape, and optionally
wherein the relief region has a relief structure that adjoins the two arches.

8. (canceled)

9. The mandibular advancement device according to claim 7, wherein the thickness of the retainer increases from a rearward position to a forward position.

10. The mandibular advancement device according to claim 9, wherein the thickness of the retainer increases such that the thickness is greatest in the region of the relief structure and apex of the arches of the support structure.

11. The mandibular advancement device according to claim 1, wherein the upper retainer forms part of an upper tray and the lower retainer forms part of a lower tray, the upper and lower trays each comprising a portion positionable over respective teeth of a user's upper and lower jaw, and optionally, wherein the lower tray is open in a front region so as to, in use, not cover a front side of teeth on a user's lower jaw and the upper tray is open in a rear region so as to, in use, not cover a rear side of teeth of a user's upper jaw, and optionally wherein upper and lower tray are connected by a hinged joint.

12. The mandibular advancement device according to claim 1, wherein, in use, the upper retainer is positioned to be substantially above the lower retainer;
wherein the upper retainer and the lower retainer each have a generally semi-oval shape, and
the lower retainer is profiled such that in use, at least a portion of the lower retainer is profiled to protrude more radially outwards from the generally semi-oval shape of the lower retainer at an end of the lower retainer furthest from the upper retainer than an end nearest the upper retainer; and/or
the upper retainer is profiled such that in use, at least a portion of the upper retainer is profiled to protrude more radially inwards from the generally semi-oval shape of the upper retainer at an end of the upper retainer furthest the lower retainer than an end nearest the lower retainer.

13. A mandibular advancement device having an upper retainer positionable in front of teeth of a user's upper jaw and connected to a lower retainer positionable behind teeth of a user's lower jaw, such that, in use, the upper retainer is positioned to be substantially above the lower retainer;
wherein the upper retainer and the lower retainer each have a generally semi-oval shape, and
the lower retainer is profiled such that in use, at least a portion of the lower retainer is profiled to protrude more radially outwards from the generally semi-oval shape of the lower retainer at an end of the lower retainer furthest from the upper retainer than an end nearest the upper retainer; and/or
the upper retainer is profiled such that in use, at least a portion of the upper retainer is profiled to protrude more radially inwards from the generally semi-oval shape of the upper retainer at an end of the upper retainer furthest the lower retainer than an end nearest the lower retainer.

14. The mandibular advancement device according to claim 13, wherein the lower retainer is curved from a position of minimal outward radial protrusion to a position of maximum outward radial protrusion; and/or the upper retainer is curved from a position of minimal inward radial protrusion to a position of maximum inward radial protrusion, or wherein the lower retainer is angled from a position of minimal outward radial protrusion to a position of maximum outward radial protrusion, and/or the upper retainer is angled from a position of minimal inward radial protrusion to a position of maximum inward radial protrusion.

15. The mandibular advancement device according to claim 13, wherein the lower retainer comprises a radially outward protruding rim; and/or upper retainer comprises a radially inward protruding rim, and optionally, wherein the rim of the lower and/or upper retainer is rounded.

16. The mandibular advancement device according to claim 13, wherein one of the lower and upper retainers is profiled to protrude at or between approximately 2 and 5 mm.

17. A mandibular advancement device having an upper retainer positionable in front of teeth of a user's upper jaw and connected to a lower retainer positionable behind teeth of a user's lower jaw, wherein
the lower retainer is profiled such that, in use, a region of the lower retainer intended to apply a force to move the jaw forwards is profiled so as to, in use, apply increased force in a location corresponding to an end of one or more teeth nearer a user's lower gum than a location corresponding to an end of one or more teeth furthest from a user's lower gum; and/or
the upper retainer is profiled such that, in use, a region of the upper retainer intended to apply a force to anchor the device in a user's mouth is profiled so as to, in use, apply increased force in a location corresponding to an end of one or more teeth nearer a user's upper gum than a location corresponding to an end of one or more teeth furthest from a user's upper gum.

18. A method of manufacturing the mandibular advancement device according to claim 1,
wherein the mandibular advancement device comprises an impression of a user's teeth, and the method comprises:
forming an impression of only the rearward teeth of a user's lower jaw, or alternatively wherein the mandibular advancement device comprises an impression of a user's teeth, and the method comprises:
forming an impression of teeth of a user's lower jaw; and
removing a portion of the impression in a forwards region of the impression to form the relief region.

19. (canceled)

20. (canceled)

21. The mandibular advancement device according to claim 12, wherein the lower retainer is curved from a position of minimal outward radial protrusion to a position of maximum outward radial protrusion; and/or the upper retainer is curved from a position of minimal inward radial protrusion to a position of maximum inward radial protrusion, or wherein the lower retainer is angled from a position of minimal outward radial protrusion to a position of maximum outward radial protrusion, and/or the upper retainer is angled from a position of minimal inward radial protrusion to a position of maximum inward radial protrusion.

22. The mandibular advancement device according to claim 12, wherein the lower retainer comprises a radially outward protruding rim; and/or upper retainer comprises a radially inward protruding rim, and optionally, wherein the rim of the lower and/or upper retainer is rounded.

23. The mandibular advancement device according to claim 12, wherein one of the lower and upper retainers is profiled to protrude at or between approximately 2 and 5 mm.
